(19) United States
(12) Patent Application Publication
(10) Pub. No.: US 2004/0166503 A1
(43) Pub. Date: Aug. 26, 2004

(54) Methods for gene expression profiling

(76) Inventors: Zhijian Chen, Elkins Park, PA (US); Hongxie Shen, Elkins Park, PA (US); Kenneth D. Tew, Plymouth Meeting, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/475,892

(22) PCT Filed: May 7, 2002
(86) PCT No.: PCT/US02/14439

(51) Int. Cl. C12Q 1/68; C12P 19/34

(52) U.S. Cl. 435/6; 435/91.2

(57) ABSTRACT

A method for detecting differentially expressed genes in a test sample is provided.

This application claims priority to U.S. provisional Application 60/289,167 filed May 7, 2001, the entire contents of which are incorporated by reference herein.

Pursuant to 35 U.S.C. §202(c) it is acknowledged that the U.S. Government has certain rights in the invention described, which was made in part with funds from the National Institutes of Health, Grant Numbers CA06927, RR05539 and CA85660.

### FIELD OF THE INVENTION

The present invention relates to the fields of gene expression and methods for analysis thereof. More specifically, methods for amplified differential gene expression (ADGE) are provided. Such methods may be coupled with microarray methodology to facilitate analysis of differential gene expression in tissue types and cells of interest.

### BACKGROUND OF THE INVENTION

Several publications and patent documents are cited throughout the specification in order to describe the state of the art to which this invention pertains. Each of these references are incorporated herein as though set forth in full.

Regulation of gene expression underlies cellular responses to a wide range of biological stimuli. Characterization of differences, both qualitative and quantitative, in transcript expression patterns provides information which is critical to understanding mechanisms underlying diverse processes such as evolutionary development, malignant transformation and stress responses. For example, drug resistant cell lines provide a biological system for evaluating biological stresses induced by various forms of anti-cancer therapies, wherein acquired resistance is achieved through the sequential selection pressure of escalating drug concentrations.

There are presently a number of methodologies that have been developed to identify altered mRNA expression in model systems. For example, these include differential display (Liang et al. (1992) Science 257:967-970; Matz et al. (1997) Nucleic Acids Res. 25:2541-2542; Kihroki et al. (1999) Biochem. Biophys. Res. Commun. 262:365-367; Wang et al. (1999) Nucleic Acids Res. 27:4609-4618), suppression substractive hybridization (SSH) (Diatchenko et al. (1996) Proc. Natl. Acad. Sci. 93:6025-6030), representational difference analysis (RDA) (Lisitsyn et al. (1994) Science 259:946-951; Hubank et al. (1994) Nucleic Acids Res. 22:5640-5648), serial analysis of gene expression (SAGE) (Zhang et al. (1997) Science 276:1268-1272; Spinella et al. (1999) Nucleic Acids Research 27:e22) and DNA microarray (Khan et al. (1999) Electrophoresis 20:223-229; Coller et al. (2000) Proc. Natl. Acad. Sci. 97:3260-3265). Among these methods, sensitivity and reliability vary, depending on their displaying systems. One consistency, however, is that these techniques do not amplify possible expression differences prior to displaying them.

### SUMMARY OF THE INVENTION

In accordance with the present invention, a novel method for assessing alterations in gene expression profiles is disclosed. Amplified differential gene expression (ADGE) is a technique designed to profile gene expression of the whole transcriptome or to compare expression of a set of genes between two samples. ADGE employs hybridization to quadratically amplify the ratio of an expressed gene between control and tester samples before displaying. The subtle structures of adapters and primers are designed for displaying the amplified ratio of an expressed gene between two samples. For certain applications, four selective nucleotides at the 3′ end of primers are used to increase PCR efficiency for targeted molecules and to improve detection of PCR products. Double PCR with the same pair of primers expands the detection range, especially for genes of low abundance. Integration of these steps makes ADGE sensitive and accurate. Us of the method to assess alterations in gene expression patterns in drug resistant human tumor cell lines showed that ADGE accurately profiled expression levels for induced, repressed or unchanged genes. The qualitative expression patterns for ADGE were verified with RT-PCR.

In yet another embodiment of the invention, the ADGE method described above is combined with microarray analysis. This approach combines the high throughput of microarray with the detection, precision, accuracy and sensitivity provided by ADGE.

### BRIEF DESCRIPTION OF THE DRAWINGS

FIG. 1. Schematic representation of ADGE. Double stranded cDNA of control and tester samples are cut with the Taq I restriction enzyme. Taq I digestion generates three types of molecules: type A with the Taq I cut site at one end, type B with the Taq I cut sites at both ends, and type C without the Taq I cut site at either end. Type B is selected as the target molecule. The Taq I fragments of control cDNA and tester DNA are ligated to CT adapter and TT adapter (sequences at the bottom), respectively. Molecules of type A and type B are ligated to adapters. The same amounts of control and tester DNA are hybridized to generate the hybrid DNA of type B, which quadratically amplifies the ratio of control and tester DNA. After filling in the ends of adapters, hybridized DNA is amplified with a pair of CT primers and with a pair of TT primers with the same selective nucleotides, respectively. CT primers amplify control type B DNA exponentially and hybrid type B DNA and control type A DNA linearly. TT primers amplify tester type B DNA exponentially and hybrid type B DNA and tester type A DNA linearly. The PCR products are separated. The bands of interest are isolated and identified. The sequences of primers CT200 and TT200 are presented as examples. The region corresponding to TaqI site is in bold and four selective nucleotides are underlined. The differences between TT primers (or CT primers) are the four selective nucleotides.

FIG. 2. ADGE profile of HL60 and HL60/ADR cell lines. The two lanes under one bracket were compared, the left lane representing HL60 DNA (control), the right lane representing HL60/ADR DNA (tester). The bands identified by arrows were isolated for sequencing. Panel A: primary PCR products, separated for 3.5 hr at 120 v on 2.5% Metaphor agarose gel containing GelStar gel stain. Panel B: secondary PCR products, separated on 3% Metaphor gel at 120 v for 3 hr.

FIG. 3. RT-PCR of A1-A6. The same total RNA samples for making hybridized DNA were reverse-transcribed with Superscript II reverse transcriptase. The number of PCR cycles was selected within the range of the linear amplification for each gene, 25 cycles for A1, 30 cycles for A2, A3 and A6, 35 cycles for A4 and A5. The annealing temperature for each pair of primers was determined based upon their Tₘ, 58° C. for A1 and A3, 60° C. for A2, 70° C. for A4 and A5, 62° C. for A6. 12 μl of each reaction was separated on 1.8% agarose gel at 100 v for 1 hr.

FIG. 4. A schematic diagram showing ADGE coupled with microarray. Total RNA is reverse-transcribed into double stranded cDNA that is cut with Taq I. The control cDNA and tester cDNA are ligated with CT adapter and TT adapter, respectively. The adapterized control and tester DNA are reassociated. The hybridized cDNA is used as PCR templates. Cy3-dCTP and Cy5-dCTP are integrated into the control and tester PCR products, respectively. The labeled PCR products are then hybridized on a microarray chip.

FIGS. 5A and 5B. A pair of MA plots obtained from conventional microarray and ADGE microarray. A is an average of log₂ Cy5 and log₂ Cy3, representing intensities of spots. M is a difference of log₂ Cy5 and log₂ Cy3, representing the expression ratios in the power of 2, with positive values for up-regulated genes, negative values for down-regulated genes and 0 for unchanged genes.

FIG. 6. A graph showing the number of genes which demonstrate a 2 fold or greater change when assessed using conventional microarray and ADGE microarray. The ratios are averages of three replicates. The “−” before ratio values indicates down-regulated genes. The values are the ratios of HL60 (Cy3) to HL60/TLK286 (Cy5). Otherwise, the values are the ratios of HL60/TLK286 (Cy5) to HL60 (Cy3).

FIGS. 7A and 7B. Analysis of gene expression profiles using ADGE microarray is very reproducible among replicates. The values on axis are the ratios of HL60/TLK286 (Cy5) to HL60 (Cy3). The correlation between replicate 1 and replicate 2 consists of 8086 data points (the top panel). The correlation between replicate 1 and replicate 3 consists of 8269 data points (the bottom panel).

FIGS. 8A and 8B. A pair of graphs showing the variances of Cy3 (HL60) (the top panel) and Cy5 (HL60/TLK286) (the bottom panel) in ADGE microarray and regular microarray. The transformed values of Cy3 and Cy5 were used to calculate the variances.

FIG. 9. A graph showing the number of genes with confidence levels of 90% or greater when assessed by either ADGE microarray or conventional microarray. The confidence level is the result of t-test for each gene.

### DETAILED DESCRIPTION OF THE INVENTION

In accordance with the present invention, a method is provided that quadratically amplifies the ratio of a gene in two samples before displaying. The quadratic amplification is achieved based on the principle of the algebra formula (a+b)(a+b)=a²+b²+2ab. The quadratic amplification keeps the same ratio for those genes with the same transcription level, but quadratically increases the ratio for these with different expression levels. By applying this amplification method to the differential display, a new technique, amplified differential gene expression (ADGE), was developed to reveal gene expression profiles between two samples. Application on two closely related cell lines indicates that ADGE is both sensitive and accurate and provides significant advantages over other technologies.

The ADGE technique also involves DNA reassociation and PCR. In an alternative embodiment of the method of the invention, a combination of the ADGE technique with DNA microarray methodology is disclosed. This combined method has been applied to assess gene profiles in a prodrug TLK286 resistant cell line. Comparative analysis of ADGE microarray with conventional microarray showed that the ADGE microarray method detects small changes of gene expression with greater sensitivity and reliability due to the quadratic magnification of expression ratios for up- and down-regulated genes Additionally, ADGE microarray methods require less starting material than conventional microarray. For example, 125 ng of total RNA was sufficient to perform ADGE microarray for one slide hybridization as compared to 20 μg of total RNA for conventional microarray, a 160 fold difference. As yet a further advantage, ADGE microarray generated stronger signals than those observed using conventional microarray methods.

The following definitions are provided to facilitate an understanding of the present invention.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specfically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield an primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “adapter” as used herein refers to short (2, 3, 4, 5, 10, 20, or 25 nucleotides) nucleotide sequences which are ligated to fragments of control and tester DNA molecules following fragmentation of the same by techniques such as restriction enzyme digestion. While CT and TT adapters are exemplified herein, the skilled artisan appreciates that adapters may be designed using any appropriate nucleotide “sets” that provide adapter function to the method.

The terms “control DNA” and “tester DNA” refer to the different nucleotide samples to be compared using the ADGE or ADGE microarray methods of the invention to assess differences in gene expression profiles therein. Exemplary samples to be compared include, without limitation, nucleic acid isolated from normal vs. diseased tissues or cells, such as normal and malignant cells or nucleic acid isolated from drug sensitive and drug resistant cells as described herein.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “substantially pure” refers to a preparation comprising at least 50-60% by weight of a given material (e.g., nucleic acid, oligonucleotide, protein, etc.). More preferably; the preparation comprises at least 75% by weight, and most preferably 90-95% by weight of the given compound. Purity is measured by methods appropriate for the given compound (e.g. chromatographic methods, agarose or polyacrylamide gel electrophoresis, BPLC analysis, and the like).

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radioimmunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like. The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other manners, the introduced nucleic acid may exist in the recipient cell or host organism only transiently.

A “cell line” is a clone of a primary cell or cell population that is capable of stable growth in vitro for many generations.

The phrase “detectable label as used herein refers to any molecule which facilitates detection and measurement of alterations in gene expression profiles. In a particularly preferred embodiment of the methods of the invention, one or more labels are attached to the sample nucleic acids. The labels may be incorporated by any of a number of means well known to those of skill in the art. However, in a preferred embodiment, the label is simultaneously incorporated during the amplification step in the preparation of the sample nucleic acids or probes. For example, polymerase chain reaction (PCR) with labeled primers or labeled nucleotides will provide a labeled amplification product. The nucleic acid (e.g., DNA) may be amplified, for example, in the presence of labeled deoxynucleotide triphosphates (dNTPs). For some applications, the amplified nucleic acid may be fragmented prior to incubation with an oligonoucleotide array, and the extent of hybridization determined by the amount of label now associated with the array. In a preferred embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Such labeling can result in the increased yield of amplification products and reduce the time required for the amplification reaction. Means of attaching labels to nucleic acids include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., see below and, e.g., Molecular Probes, Eugene, Oreg., USA), radiolabels (e.g., ³² P, ³³ P, ³⁵ S, ¹²⁵ I and the like), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, which are incorporated in their entirety by reference herein.

Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy3 and Cy5, macrocyclic chelates of lanthamide ions, e.g. quantum dye™, fluorescent energy transfer dyes, such as thiazole orange-ethidium heterodimer, TOTAB, etc. As mentioned above, labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. For each sample of RNA, one can generate labeled oligos with the same labels.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another utilizing the methods of the present invention.

Suitable chromogens which may be employed include those molecules and compounds which absorb light in a distinctive range of wavelengths so that a color can be observed or, alternatively, which emit light when irradiated with radiation of a particular wave length or wave length range, e.g., fluorescers.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectible signal or donates energy to a fluorescent acceptor. A diverse number of families of compounds have been found to provide chemiluminescence under a variety or conditions. One family of compounds is 2,3-dihydro-1,4-phthalazinedione. The must popular compound is luminol, which is the 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence can also be obtained with oxalates, usually oxalyl active esters, e.g., p-nitrophenyl and a peroxide, e.g., hydrogen peroxide, under basic conditions. Alternatively, luciferins can be used in conjunction with luciferase or lucigenins to provide bioluminescence.

### Uses of ADGE and ADGE Coupled with Microarray

The ADGE and ADGE microarray methods of the invention may be used to advantage to assess and measure the presence and expression of individual genes or entire genomes in nucleic acid samples isolated target tissues or cells of interest. The present methods provide significant advantages over conventional differential display and microarray methods. Specifically, the ADGE microarray method of the invention provides greater detection sensitivity, improved accuracy and reliability and enables the investigator to use less starting materials than the prior art methods. Differences in gene expression may be assessed in cell types, including without limitation, differences between diseased and normal cells, drug sensitive and drug resistance cells, and malignant and normal cells.

The following examples are provided to illustrate embodiments of the invention. They are not intended to limit the invention in any way.

### EXAMPLE I

### Amplified Differential Gene Expression (ADGE)

The following materials and methods are provided to facilitate the practice of Example I.

Cell lines

The HL60/ADR cell line is resistant to adriamycin (ADR) and derived from the wild type HL60 by step wise selection. Both HL60/ADR and HL60 cells were cultured in RPMI-1640 medium supplemented with 2 mM L-glutamine, 50 U/ml penicillin, 50 μg/ml streptomycin and 10% heat-inactivated fetal bovine serum. Cells were maintained in a humidified atmosphere of 5% CO₂ at 37° C.

Adapters and primers

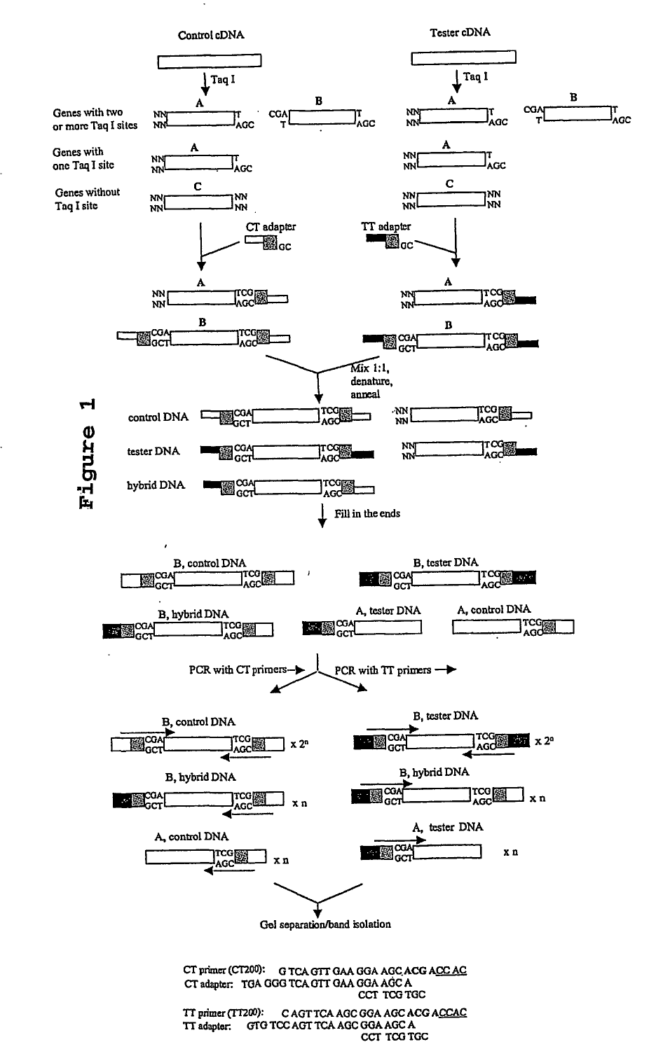

The adapter CT is for control and adapter TT for tester sample. Both have cohesive ends complementary to Taq I (FIG. 1). Primer CT is complementary to adapter CT while primer TT is complementary to adapter TT. The assigned number in the primer name represents the four 3′ end nucleotides selective for a set of displayed genes. The selective nucleotides for primers used in this experiment are iCT22/TT22: TGAT, CT60/TT60: ATTC, CT134/TT134: AGTC, CT193/TT193: GGAG, CT196/TT196: GCAC, CT200/TT200: CCAC, CT218/TT218: GGCT.

Preparation of hybridized DNA

Total RNA was isolated from HL60 and HL60/ADR cells with a Qiagen RNeasy Midi Kit. 5.5 μg total RNA of HL60 and HL60/ADR were reverse-transcribed to cDNA using the GIBCO cDNA Synthesis System. After phenol extraction and ethanol precipitation, cDNA samples were resuspended in 30 μl of ddH₂O, cut with 40 units of Taq I in a 40 μl reaction at 65° C. for 2 hours. Since the HL60 cell line was used as control, Taq I fragments of HL60 DNA were ligated to CT adapter. The Taq I fragments of HL60/ADR DNA were ligated to TT adapter. Ligation reactions were set up by adding 5 μl 10×SB buffer, 4 μl 30 μM adapter, 3 units of ligase to 40 μl of restriction reaction and carried out overnight at 14° C. 15 μl of HL60 ligation mixtures and 15 μl of HL60/ADR ligation mixtures were mixed with 30 Ill of 2×HB buffer and hybridized at 95° C. for 5 minutes, then 68° C. for 10 hours. The hybridized DNA was used directly as template for PCR amplification.

PCR Amplification of Hybridized DNA

The hybridized DNA was amplified with a pair of CT primers complementary to the CT adapter and with a pair of TT primers with the same selective nucleotides as CT primers. Clontech cDNA polymerase was used in a 50 Ill reaction consisting of 1 μl of hybridized DNA, 1 μl of each primer, 1 μl of 10 mM dNTPS, 5 μl of 10× buffer, 1 μl of polymerase and 40 μl of H₂O. The reaction cycling conditions were 72° C. for 5 minutes (for filling in adapter ends), 94° C. for 1 minute, then 30 cycles of 94° C. for 30 seconds, 70° C. for 30 seconds, 72° C. for 40 seconds, then 72° C. for 5 minutes for a final extension. 1 μl of primary PCR product was taken to make up 50 μl of secondary PCR reaction with the same primers for the primary PCR. The cycling conditions were 94° C. for 1 minute, then 14 cycles of 94° C. for 30 seconds, 70° C. for 30 seconds, 72° C. for 40 seconds, then 72° C. for 5 minutes.

Analysis of differentially expressed genes

The primary PCR product was separated for 3.5 hr at 120 v on 2.5% Metaphor gel (FMC, Rockland, Minn.) containing GelStar gel stain (FMC). The secondary PCR product was separated on 3% Metaphor gel for 3 hr at 120 v. By comparing a pair of CT primers with a pair of TT primers with the same selective nucleotides, the bands of interest were identified, isolated and suspended in 30 μl of EB buffer. DNA was eluted by incubating the isolated band at 60° C. for 15 minutes. The eluates of these bands were reamplified with the corresponding primers. The reamplification reaction consisted of 1 μl of elute, 1 μl of each primer, 1 μl of 10 mM dNTPS, 5 μl of 10× buffer, 1 μl of Clontech cDNA polymerase and 40 μl of H₂O. The reaction cycling conditions were 94° C. for 1 minute, then 30 cycles of 94° C. for 30 seconds, 70° C. for 30 seconds, 72° C. for 40 seconds, then 72° C. for 5 minutes. The PCR products were gel-purified and sequenced with the corresponding primers. Sequences were analyzed with software Sequencher 3.1 and searched against GenBank.

RT-PCR

RT-PCR is used to verify the ADGE results. Primers for RT-PCR were designed based upon the sequences from the identified genes or fragments with software OLIGO 4.0. The same total RNA samples that were used for making hybridized DNA were reverse-transcribed with Superscript II reverse transcriptase. The cDNA was used as the template for PCR amplification. After different PCR cycles were tested, specific PCR cycle conditions were selected within the range of the linear amplification for each gene, to optimize the levels of differential expression; i.e. 25 cycles for A1, 30 cycles for A2, A3 and A6, 35 cycles for A4 and A5. The annealing temperature for each pair of primers was determined based upon their Tₘ, 58° C. for A1 and A3, 60° C. for A2, 70° C. for A4 and A5, 62° C. for A6. 12 μl of each reaction was separated on 1.8% agarose gel at 100 v for 1 hr.

### RESULTS

The scheme of the ADGE technique is shown in FIG. 1. Two different nucleic acid samples are selected, one for control, the other for tester. After they are synthesized from total RNA or mRNA, the control and tester cDNA are cut with Taq L The Taq I fragments of control DNA and tester DNA are ligated to CT adapter and TT adapter, respectively. The same amount of adapterized control and tester DNA are hybridized together. The hybridized DNA is amplified with a pair of CT primers complementary to CT adapter and with a pair of TT primers with the same selective nucleotides, respectively. A secondary PCR reaction is set up with the same pair of primers after primary PCR. The primary and secondary PCR products are separated on gels. By comparing pairs of CT and TT primers with the same selective nucleotides, unique bands are isolated and identified. Finally, elutes of these bands are reamplified and sequenced. The corresponding genes are searched against a database such as GenBank.

The major advantage of ADGE is that it quadratically amplifies the ratio of a gene in two samples before displaying. The quadratic amplification is achieved through hybridization of control and tester DNA. As shown in FIG. 1, ADGE requires that the adapterized control DNA and the adapterized tester DNA be mixed together at a ratio of 1:1 in weight or molarity. After denaturing and annealing, three different types of the targeted molecule type B are formed: control DNA with CT adapters on both ends, tester DNA with TT adapters on both ends, hybrid DNA with the CT adapters on one end and the TT adapters on the other end. The relative amount of the three types of targeted molecules for each gene is in theory governed by the algebra formula

(a+b)(a′+b′)=aa′+bb′+a′b+ab′

where

a—the number of sense strands of one gene in the control sample

b—the number of sense strands of the gene in the tester sample

a′—the number of antisense strands of one gene in the control sample

b′—the number of antisense strands of the gene in the tester sample

aa′—the number of double strands of the gene in the control sample

bb′—the number of double strands of the gene in the tester sample

a′b, ab′—the number of double strands of the hybridized DNA.

Therefore, for a gene with the same transcript level between control and tester samples, the ratio of aa′ and bb′ stays the same after denaturing and annealing. However, fifty percent of the nucleotides are in the form of hybrid a′b or ab′, which normalizes the abundant house-keeping genes. For a gene with a different expression level between control and tester samples, the ratio of aa′ and bb′ is quadratically amplified after denaturing and annealing. For example, for a gene overexpressed 5 times in tester over control, bb′/aa′=5. Thus the formula is

(a+5b)(a′+5b′)=aa′+25bb′+5ab′+5a′b.

After denaturing and annealing, the ratio of bb′/aa′ increases from 5 to 25. If expression of another gene is, for example, reduced 10 times in tester, then the formula is

(10a+b)(10a′+b′)=100aa′+bb′+10ab′+10a′b.

Therefore, the ratio of aa′ to bb′ increased from 10 to 100 after denaturing and annealing.

The structure of the adapter and primer is critical to ensure the quadratic amplification of a gene ratio after hybridization. The adapters are composed of long and short oligos (FIG. 1). The short oligos are the same between CT and TT adapters in order to form hybrid DNA molecules. The optimum complementary region is 7 nucleotides. If it is too short, the adapters may not be stable, if too long, cross priming becomes possible. The adapters have cohesive ends complementary to TaqI. However, the TaqI site is not recovered from ligation. The CT and TT primers consist of regions complementary to the adapters, regions corresponding to TaqI sites and four selective nucleotides. The length of different regions between CT and TT primers should be sufficient to prevent cross priming (at least ten nucleotides long). Although their sequences can be changed, CT, TT adapters and primers are designed using the recommended principle so that CT primers amplify aa′ molecules exponentially and a′b and ab′ molecules linearly while TT primers amplify bb′ molecules exponentially and a′b and ab′ molecules linearly.

The ADGE scheme in FIG. 1 can be adapted by implementing a number of modifications. For example, other four base pair restriction enzymes besides Taq I can be used and are recommended for those genes with one Taq I site or without a Taq I site. The 3′ end of CT and TT adapters can be phosphorylated. The number of the selective nucleotides can be less than 4, in which case, a sequencing gel system is recommended to separate PCR products. The PCR products can be visualized with either isotope or fluorescence labels.

Preferably, four selective nucleotides are used to reduce the number of candidate PCR templates, thereby increasing the PCR efficiency for each candidate template and allowing the use of small gels to separate PCR products. A pair of primers with four selective nucleotides at the 3′ ends amplifies 1/65,536 of the total genes in a transcriptome at one time. Assuming 50,000 genes in a transcriptome and an average gene size of 1500 bp theoretically generating 5 Taq I fragments of type B, there are 250,000 Taq I fragments of type B amplifiable with PCR. Therefore, every PCR produces an average of 3.8 bands (250,000/65,536). Amplifying such a small set of genes at a time increases the PCR efficiency for targeted molecules. Additionally, it is preferred that high resolution Metaphor agarose gels and high sensitivity GelStar gel stain be used to detect the small set of PCR products. These factors also facilitate a more efficient and safer methodology.

In cases where rare genes are being detected, it is recommended that a double PCR be used. If 2% of total RNA is poly(A) RNA and average gene size is 1500 bp, 5 μg of total RNA can generate 0.40 pmole of cDNA. If 0.4 pmole of control DNA is hybridized to 0.4 pmole of tester DNA in 60 μl and 50,000 genes are in a transcriptome, 1 μl of hybridized DNA for PCR contains 0.8/(50,000×60)=2.7×10⁷ pmoles for an intermediately prevalent gene. Such low amounts of template, while enough for abundant genes, are not sufficient to detect products of a single primary PCR for a gene of intermediate or low expression level. Thus, double PCR for each pair of primers is recommended. The primary PCR products will identify abundant genes, while the secondary PCR products detect genes of low abundance. Therefore, double PCR increases the sensitivity and detection range of ADGE.

Differential Gene Expression Between HL60 and HL60/ADR

The ADGE technique can be applied in two modes: globally screening the whole transcriptome with randomly selected primers; or partially screening a set of known genes with targeted primers. DNA-PK and MRP are known to be overexpressed in HL60/ADR cells (S hen et al. (1998) Biochim, Biophys. Acta. 1381:131-138). From the Taq Irestriction maps of DNA-PK and MRP, a 610 bp DNA-PK fragment (8052-8661 bp) and a 555 bp MRP fragment (3719-4274 bp) were selected. Based on the internal sequences next to Taq I sites, primers CT200, CT218, TT200, TT218 were designed for DNA-PK; primers CT193, CT22, TT193, TT22 for MRP. The primers CT196, CT134, TT196, TT134 were also designed for amplifying the segment from 285 bp to 741 bp of the β-actin gene which was used as internal control.

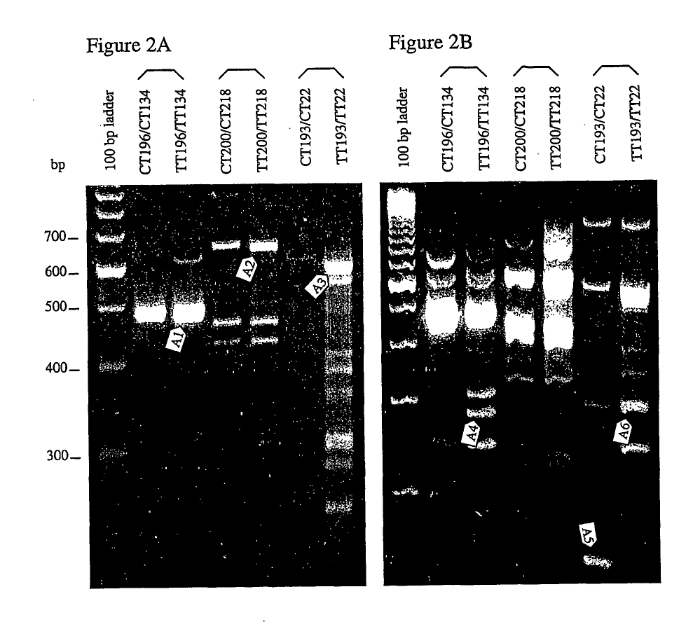

FIG. 2 shows the ADGE profile of primary PCR (Panel A) and secondary PCR (Panel B). Band A1 was identified as a fragment of the β-actin gene. Bands A2 and A3 were identified as fragments of DNA-PK and MRP, respectively. The secondary PCR in Panel B displayed other novel bands A4-A6. The sequences identified A4 as novel, A5 as seb4B and A6 as DnaJ (Table 1).

TABLE 1

Summary of sequence analysis of bands A1-A6. Arrow indicates the mis [symbol]
between selective nucleotides of primer and gene sequence adjacent to
the Taq I si

[symbol]

Sequences of two ends and

| | | | |
|---|---|---|---|
| GenBank | Gene | Position of two ends | selective nucleotides |

| | | | | | | |
|---|---|---|---|---|---|---|
| Bands | Genes | Accession | size | 5′ end | 3′ end | of primer |

| | | | | | | |
|---|---|---|---|---|---|---|
| | | | | | | CT196--GCAC |
| A1 | actin | NM-001101 | 1793bp | 285bp | 741bp | TCGAGCAC--//--GACTTC [symbol] |
| | | | | | | TT200--CCAC CTGA--C [symbol] |
| A2 | DNA-PK | HSU34994 | 12780bp | 8052bp | | TCGACCAC--//--AGCCTC [symbol] |
| | | | | | | TCGG--T [symbol] |
| | | | | | | TT193--GGAG |
| A3 | MRP | L05628 | 5011bp | 3719bp | 4274bp | TCGAGGAG--//--ATCATC [symbol] |
| | | | | | | TAGT--T [symbol] |
| | | | | | | TT134--AGTC |
| A4 | novel | | | | | TCGAAGTC--//--GTGCTC [symbol] |
| | | | | | | CACG--T [symbol] |
| A5 | seb4B | X75315 | 1438bp | 144bp | 268bp | CT193--GGAG |
| | | | | | | TCGAGGAG--//--ATCATC [symbol] |
| | | | | | | TAGT--C [symbol] |
| | | | | | | TT193--GGAG |
| A6 | DnaJ | D13388 | 1435bp | 873bp | | TCGACGAG--//--ATCATC [symbol] |
| | | | | | | ↑ TAGT-- [symbol] |

Arrow indicates the mismatch between selective nucleotides of primer and gene sequence adjacent to the TaqI site.

Because PCR in ADGE has several types of templates with different initial concentrations while RT-PCR has a single type of template with concentrations different from those in ADGE, the PCR efficiency differs between them. A measure of the comparative quantitative relationship between ADGE and RT-PCR is provided by Table 2.

TABLE 2
The ratios of HL60/ADR to HL60 for bands
A1-A6 from ADGE and RT-PCR

| Bands | A1 | A2 | A3 | A4 | A5 | A6 |
|---|---|---|---|---|---|---|
| ADGE | 1.37 (1) | 3.79 (2.76) | 47.6 (34.7) | 13.5 (9.85) | 0.027 (0.02) | 4.07 (2.97) |
| RT-PCR | 1.07(1) | 2.23 (2.08) | 9.81 (9.16) | 1.34 (1.25) | 0.86 (0.80) | 1.53 (1.43) |

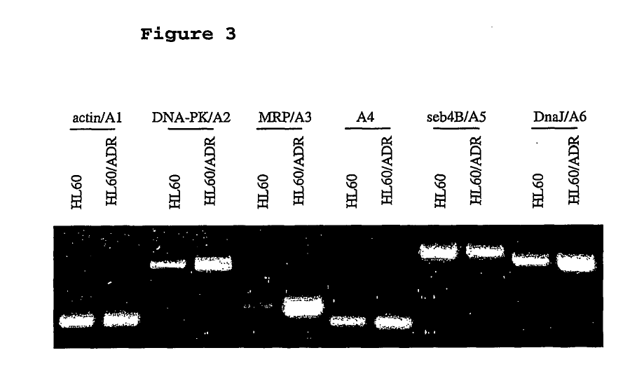

The ADGE of A1, A2 and A3 were from quantifying the corresponding bands in FIG. 2 with NIH Image 1.62. The ADGE data of A4, A5 and A6 were from bands in FIG. 2. The RT-PCR data of A1-A6 were from the bands in FIG. 3. The data in parentheses are normalized with the A1 (beta-actin) data in ADGE and RT-PCR.

The ratios of HL60/ADR to HL60 bands were greater for the induced genes (A2, A3, A4 and A6) in ADGE than in RT-PCR. The ratios of HL60/ADR to HL60 bands decreased for the repressed gene (A1) in ADGE (Table 2). It is indicated that ADGE successfully amplified the ratio of HL60/ADR to HL60 bands for induced genes and the ratio of HL60 to HL60/ADR bands for repressed genes even though the specific amplification values showed some variation. Therefore, the ADGE technique increased the sensitivity and accuracy of detection.

The number of RT-PCR cycles was optimized for each gene, 25 cycles for actin, 30 cycles for DNA-PK, MRP and DnaJ, 35 cycles for A4 and seb4B. Therefore, actin was the most abundant of the six genes. A4 and seb4B were less abundant than DNA-PK, MRP and DanJ in the transcriptome. For these six genes, the sizes varied from 1.4 kb (DnaJ) to 12 kb (DNA-PK). The targeted positions could be the 3′ region (seb4B) or 5′ region (DnaJ) (Table 1). Analysis of end sequences showed only one mismatch between the selective nucleotides and internal sequences of genes over 12 pairs of primers (Table 1). Occurrence of the nucleotide mismatch primarily increases the number of displayed bands. For example, DnaJ should be amplified with the primer TT197 of selective nucleotides CGAG, but not with TT193 of GGAG. Since the amplifiable templates for CT193/CT22 were low or missing in HL60 cells, the PCR yield of CT193/CT22 was repeatedly shown to be lower than that of TT193/TT22 (FIG. 2).

In summary, the ADGE technique increased the sensitivity, accuracy and range of detection. It accurately profiled the expression patterns for overexpressed, repressed or unchanged genes.

### EXAMPLE II

### Coupling ADGE with Microarray

The data presented in Example I represent one application of ADGE, where four selective nucleotides are used at 3′ ends of primers and horizontal agarose gels are used as the displaying system. This approach is suited for investigating a few families of targeted genes, but is not ideal for screening the entire complexity of expressed genes in a cell line (transcriptome) as the throughput is quite low. In another embodiment of ADGE, two or three selective nucleotides are used at 3′ end of primers and the sequencing gel is used for the displaying system. The throughput is still very low since 240 PCR reactions are needed to screen the whole transcriptome for 2 selective nucleotides. However, when microarray is used as displaying system for ADGE, the throughput is very high with the need of only one chip hybridization to detect all genes spotted on the chip.

Gene expression profiles represent the signatures of cells at a specific state, providing a source of information for identifying genes that are involved in the maintenance of that state. DNA microarray technologies are designed to reveal gene expression profiles by detecting the expression levels of large numbers of genes simultaneously (Schena et al, (1995) Science, 270:467470; Lockhart and Winzeler, (2000) Nature, 405:827-836). Microarray technologies have been used to profile differential gene expression in an antioxidant responsive system (Li et al, (2002) Biological Chemistry, 277(1):388-394), and in different tumor stages (Ramaswamy et al, (2001) PNAS, 98(26):15149-15154). However, this hybridization based approach has certain drawbacks when assessing genes demonstrating only slight alterations in expression levels. Furthermore low accuracy and potentially high experimental errors have been reported. (Yue et al, (2001) Nucleic Acid Research, 29(8): e41). Finally, most microarray based methods require large amounts of biological starting material.

The ADGE technique described in Example I was designed to quadratically magnify the ratios of genes with the integration of DNA reassociation and PCR. DNA reassociation is a procedure where the control and tester DNA are mixed in the ratio of 1:1, denatured and annealed together. DNA reassociation results in the quadratic magnification of expression ratios for the up- and down-regulated genes in control and tester samples. Subsequent PCR is used to separate control DNA from tester DNA and amplifies the products thereof. The present example provides a method ADGE combined with DNA microarray (hereafter called ADGE microarray) and has been applied to assess differential gene expression in a prodrug TLK286 resistance cell line.

The prodrug TLK286 is activated by glutathione S-transferases (GST) P1-1 and A1-1 isoforms and generates the actual alkylating moieties that react with cellular nucleophiles (Gate and Tew, (2001) Expert Opin. Ther. Targets, 5(4):477-489). TLK286 is cytotoxic to a wide variety of cancer cell lines and tumors. TLK286 antitumor activity has also been observed in vivo in murine xenografts of M7609 expressing different levels of GST P1-1. Responses to this prodrug were positively correlated with the expression levels of GST P1-1. In addition, the analysis of the myelosuppressive effect of this compound demonstrated that is only mildly toxic to bone marrow stem cells and peripheral white blood cell. In transfected NIH3T3 cell lines, resistance to TLK286 was associated with the overexpression of γGCS and MRP and was partially reversed by the overexpression of GSTP1-1. Interestingly, in a resistant HL60 human promyelocytic cell line selected by chronic exposure to increasing concentrations of TLK286, a 2-fold decrease of GST P1-I expression but no increases in γGCS and MRP levels were observed. These data suggest that these two glutathione related genes are not the major mechanisms involved in the acquired resistance to TLK286 in this cell line. However, the inhibition of glutathione synthesis by BSO increases the sensitivity of the resistant cells to TLK286, further implicating the involvement of GSH in the resistance to this molecule.

The following materials and methods are provided to facilitate the practice of Example II.

Cell lines

The BL60/TLK286 cell line is resistant to the prodrug TLK286 [gamma-glutamyl-alpha-amino-beta(2-ethyl-N,N,N′,N-tetrakis(2-chloroethyl)phosphorodiamidate)-sulfonyl-propionyl-(R)-(−)phenylglycine] and is derived from the wild-type HL60 by stepwise selection. Both HL60/TLK286 and HL60 cell lines were cultured in RPMI-1640 medium supplemented with 2 mM L-glutamine, 50 U/ml penicillin, 50 μg/ml streptomycin and 10% heat-inactivated fetal bovine serum. Cells were maintained in a humidified atmosphere of 5% CO₂ at 37° C.

Conventional DNA microarray

Total RNA was isolated from both cell lines with a Qiagen RNeasy Midi kit. The conventional microarray experiment was done following the manufacturer's instructions on the FairPlay™ microarray labeling kit (Stratagene, La Jolla, Calif.). Twenty μg of total RNA from HL60 or HL60/TLK286 cells was reverse-transcribed into single stranded cDNA. The cDNA was purified with ethanol precipitation and suspended in 5 μl of 2× coupling buffer. One pack of FluoroLink™ Cy3 or Cy5 monofuctional dye (Amersham Pharmacia) was suspended in 45 μl of DMSO. Five μl of Cy3 dye was added to HL60 cDNA while 5 μl of Cy5 dye was added to HL60/TLK286 cDNA. The mixtures were incubated for 30 minutes at room temperature in the dark. The labeled HL60 cDNA and HL60/TLK286 cDNA were combined and purified. The labeled cDNA was mixed with 1.5 μl of 10 μg/μl Cot-1 DNA, 1.5 μl of 8 μg/l poly d(A), 1.5 μl of 4 μg/μl yeast tRNA, 4.5 μl of 20×SSC and 0.75 μl of 10% SDS, heated at 99° C. for 2 minutes, and then incubated at 45° C. for 20 minutes. The labeled DNA was loaded onto a microarray chip. The hybridization chamber was assembled with the microarray chip and submerged in a water bath at 63° C. for 18 hours. The microarray chip was washed in wash buffer I of 2×SSC and 0.1% SDS for 5 minutes, then in wash buffer II of 1×SSC for 5 minutes, then in wash buffer III of 0.2×SSC for 5 minutes. The slide was dried by centrifuging at 650 rpm for 5 minutes and scanned with Affymetrix 428 Array Scanner using the Cy3 and Cy5 channels. Three replicates have been done on first set of human microarray chips containing 10,368 genes each made in the Fox Chase Cancer Center Microarray Facility.

ADGE microarray

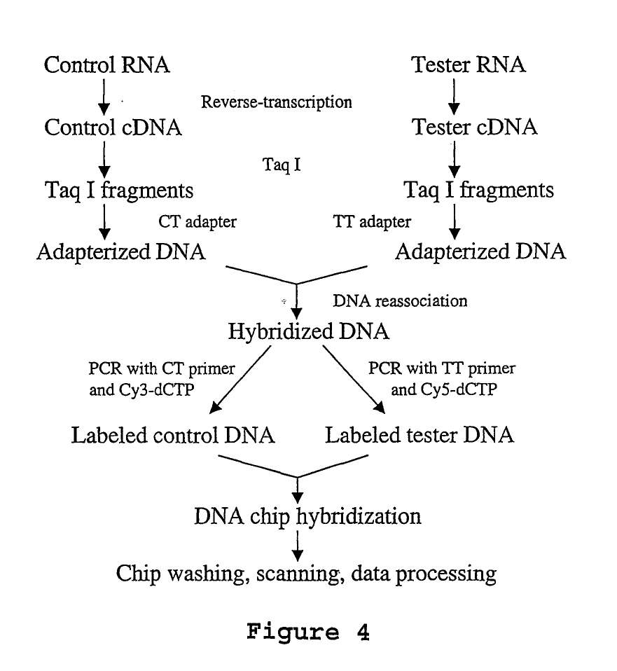

The ADGE microarray procedure was implemented following the scheme in FIG. 4. Ten μg of total RNA was reverse-transcribed into first stranded cDNA with oligo(dT)₁₂₋₁₈. Then the double stranded cDNA for HL60 and HL60/TLK286 was generated with the cDNA Synthesis System (Life Technologies, Rockville, Md.). After phenol extraction and ethanol precipitation, the cDNA was resuspended in 25 μl of ddH₂O. Both sets of cDNA were cut in 30 μl of reactions with 3 μl (30 units) of the restriction enzyme Taq I. The Taq I fragments of HL60 cDNA were ligated with the CT adapter at 14° C. for overnight with 3 μl (9 units) of T4 ligase (Promega,) while the Taq I fragments of HL60/TLK286 cDNA were ligated with the TT adapter. The adapterized HL60 cDNA and HL60/TLK286 cDNA was mixed in equivalent amounts in 30 μl of 2×HB buffer, denatured at 95° C. for 5 minutes and annealed at 68° C. for 10 hours. The reassociated DNA was used as a template for the PCR reaction. The CT primer and TT primer were complementary to the CT adapter and TT adapter, respectively. To generate the probe of HL60 DNA, a PCR reaction was set up with 0.5 μl of the reassociated DNA, 5 P1 of 10× Clontech PCR buffer, 1 μl of dNTPS (10 mM dATP, dTTP, and dGTP each, 6 mM dCTP), 4 μl of FluoroLink Cy3-dCTP (Amersham Pharmacia), 4.5 μl of 10 μM CT primer, 1 μl of Clontech cDNA polymerase and 34 μl of ddH₂O. In the PCR reaction for the probe of HL60/TLK286 DNA, Cy5-dCTP and TT primer were used instead of Cy3-dCTP and CT primer. The reaction cycling conditions were 72° C. for 5 min (for filling in the adapter ends), 94° C. for 1 min, then 30 cycles of 94° C. for 30 s, 62° C. for 30 s, 72° C. for 60 s, then 72° C. for a final extension. Three PCR reactions were set up for each probe. The Cy3 (HL60) and Cy5 (HL60/TLK286) PCR products were purified with Qiagen PCR purification kit, reduced to a final volume of 7.5 μl, mixed with 3.75 μl of 20×SSC, 0.75 μl of 10% SDS, 1.5 μl of 1 μg/μl salmon DNA and 1.5 μl of 50× Denhardt's solution, denatured at 95° C. for 5 minutes, cooled on ice and incubated at 42° C. for 15 minutes. The denatured Cy3 (HL60) and Cy5 (HL60/TLK286) DNA was mixed and loaded onto a microarray chip. The hybridization temperature and washing conditions were the same as the regular microarray. Three replicates have been done on first set and second set of human microarray chips containing 10,368 genes each.

Analysis of Microarray Data

The spots of microarray pictures were quantified with ImaGene4.1 (Biodiscovery, Los Angeles, Calif.). The Cy3 and Cy5 data of spots were integrated into a data set and transformed with GeneSight3.0 (Biodiscovery) in the following sequence: local background correction, removal of flagged spots and weak spots (less than 200 units of intensity), logarithm of base 2, ratio calculation and linear regression normalization. The transformed data were exported into Microsoft Excel. The three replicates were combined and MA plots were constructed (Tseng et al, (2001) Nucleic Acids Research, 29(12):2549-2557; Yang et al, (2002) Nucleic Acids Research, 30(4):e15).

M=log₂(Cy5/Cy3); A=log₂ (\sqrt{(Cy5*Cy3)}). After mathematical transformation, M actually is the difference between log₂(Cy5) and log₂(Cy3); A is an average of log₂(Cy5) and log₂(Cy3). M represents the ratios in the power of 2, with positive values for up-regulated genes (Cy5/Cy3), negative values for down-regulated genes (Cy3/Cy5), zero for unchanged genes. Up- and down-reuglated genes were selected based on a threshold of M values. In addition, variances of Cy3 and Cy5, t value and confidence level were calculated for each gene. Genes demonstrating significant changes in expression levels were selected based on the threshold of t values and confidence levels. The correlation between replicates was calculated for ADGE microarray.

Real Time PCR

Real time PCR was carried out in optical tubes using gene specific primers and fluorogenic probes on Smartcycler (Cepheid, Sunnyvale, Calif.). The templates of HL60 and HL60/TLK286 were first normalized with beta-actin. The internal controls of serial dilutions and replicates were used to measure the Ct values of HL60 and HL60/TLK286 for each selected gene. Ratios of changes were estimated based on the Ct values of samples and internal controls

### RESULTS

The combination of ADGE and DNA microarray

ADGE combined with DNA microarray entails labeling ADGE PCR products with dyes followed by hybridization of such products onto a microarray chip. Several alternative embodiments of the method exist. FIG. 4 shows one approach for coupling ADGE and DNA microarray. Cy3-dCTP is incorporated into control DNA and Cy5-dCTP is incorporated into tester DNA during the PCR amplification of the hybridized DNA templates. The Cy dyes can be flipped if necessary. The labeled control and tester DNA are hybridized onto a microarray chip. Other potential approaches include using aminoallyl-dUTP or biotinated dCTP. Aminoallyl-dUTP is first incorporated into control and tester PCR products that are in turn coupled with Cy dyes; Biotinated dCTP is first incorporated into control and tester PCR products that are in turn coupled with Streptavidin conjugated dyes (Motorola Life Science, Northbrook, Ill.). It is also possible to adapt other methods of signal enhancement to the coupling procedure. For example, the 3DNA fluorescent dendrimer probes (Genisphere, Montvale, N.J.) can be coupled to the CT and TT primers which then are used to amplify the control and tester DNA.

ADGE Microarray Improves Detection Sensitivity

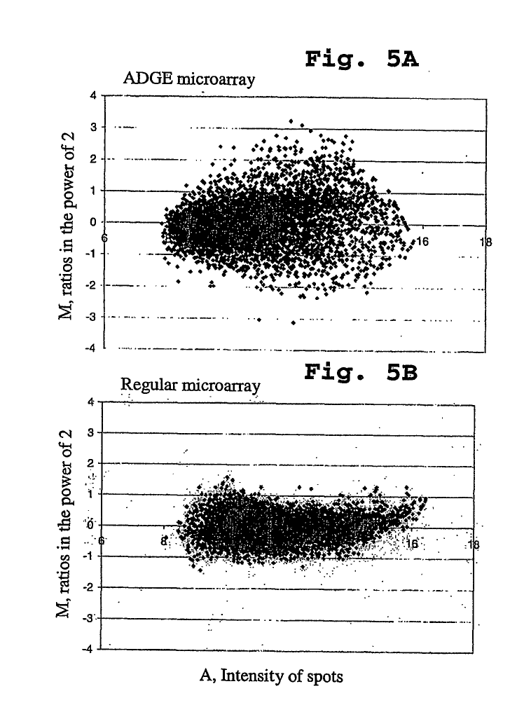

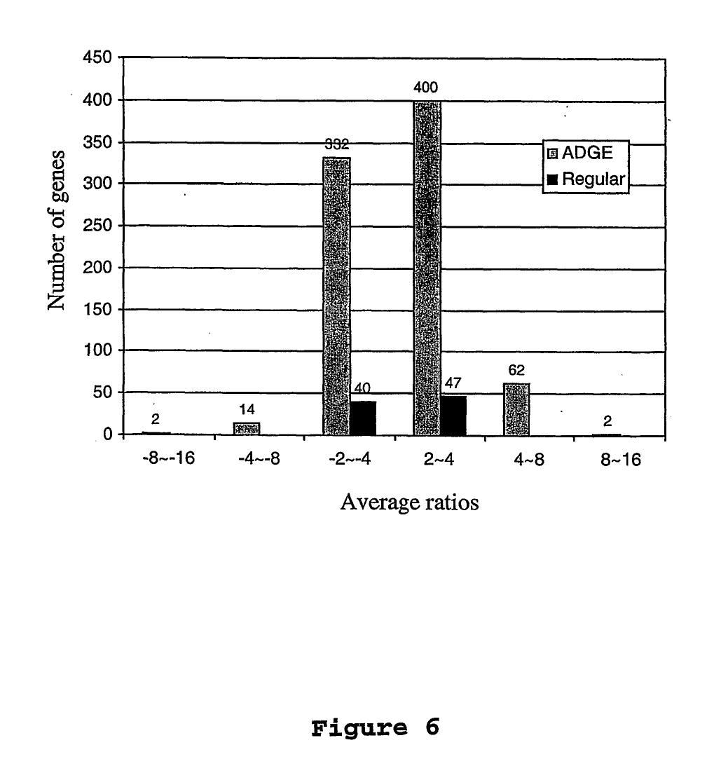

ADGE microarray magnifies expression ratios for up- and down-regulated genes. This magnification of the expression ratios makes it possible to detect small changes in gene expression. The MA plot of ADGE microarray has wider upward and downward distribution from the central area than that of conventional microarray (FIG. 5). Assessment of the overall changes of expression ratios for up- and down-regulated genes were facilitated by ADGE magnification. FIG. 6 lists the number of genes with changes of 2 fold or greater detected with ADGE microarray and conventional microarray. There were 64 genes of 4 fold or greater increase and 16 genes of 4 fold or greater decrease with ADGE microarray while there was no such alterations in these gene expression profiles were detected with conventional microarray techniques. 732 genes were identified with 2-4 fold changes in gene expression levels with ADGE microarray as compared to 87 such genes with convential microarray. Thus, alterations in the expression ratios of genes, both up-regulation and down regulation, were magnified and detected using the ADGE microarray method of the invention.

The magnification of signal achieved using ADGE microarray facilitates detection of both slight and large alterations in gene expression levels. Table 3 lists all genes with confidence level of 99% and t values of 7 or greater. 77% of these genes demonstate the same direction of expression alterations between ADGE microarray and conventional microarray, however smaller magnitudes of differenences are observed when conventional microarray is employed. 6 genes were selected for real time PCR. Five of them confirmed the results observed using either ADGE microarray or conventional microarray. For the inconsistent gene, the real time PCR result supported the results obtained when ADGE microarray was used. Therefore, the magnification achieved using the ADGE microarray method of the invention facilitates analysis of gene expression alterations, thus improving detection sensitivity.

TABLE 3
Ratios detected with ADGE microarray, regular microarray and
real time PCR for genes with confidence level of 99% and t
values of 7 or greater.

| | | | | | | Real |
|---|---|---|---|---|---|---|
| | | Confi- | | Conven- | Consis- | Time |
| Accesss No | t-value | dence(%) | ADGE | tional | tence | PCR |
| T67440 | 29.20 | 99.85 | 4.04 | 1.38 | 1** | |
| AA432106 | 16.32 | 99.31 | 3.63 | −1.48* | −1 | |
| H99257 | 15.64 | 99.93 | 5.30 | 1.75 | 1 | |
| AA425160 | 12.55 | 99.36 | 4.07 | 1.66 | 1 | 1.3 |
| W72816 | 12.10 | 99.85 | 4.05 | 1.58 | 1 | |
| H56918 | 11.31 | 99.19 | 8.18 | 1.08 | 1 | |
| AA150828 | 10.30 | 99.42 | 3.59 | −1.40 | −1 | 1.2 |
| AA890663 | 10.09 | 99.03 | 7.59 | 1.63 | 1 | 1.5 |
| N55480 | 9.64 | 99.73 | 4.82 | 1.31 | 1 | |
| AA169151 | 9.54 | 99.75 | 5.05 | 1.08 | 1 | 1.7 |
| H56918 | 9.27 | 99.62 | 6.30 | 1.45 | 1 | 2.0 |
| H98856 | 9.01 | 99.83 | 6.67 | 1.85 | 1 | |
| AA701933 | 8.56 | 99.17 | 7.10 | −1.43 | −1 | |
| W86660 | 8.41 | 99.88 | 4.11 | −1.15 | −1 | |
| R01682 | 8.11 | 99.72 | 3.94 | 1.53 | 1 | |
| N52373 | 7.78 | 99.18 | 6.09 | 1.57 | 1 | |
| AA916413 | 7.77 | 99.44 | 4.57 | −1.18 | −1 | |
| T87139 | 7.37 | 99.25 | 7.21 | 1.62 | 1 | |
| AA047567 | 7.11 | 99.20 | 4.95 | −1.08 | −1 | |
| H72119 | 7.09 | 99.08 | 10.22 | 1.49 | 1 | |
| H80708 | −8.83 | 99.03 | −4.11 | −1.44 | 1 | −1.3 |
| H83116 | −9.084 | 99.09 | −4.02 | 1.10 | −1 | |
| H73237 | −9.27 | 99.03 | −9.61 | −2.93 | 1 | |
| N55520 | −12.24 | 99.48 | −5.54 | −1.40 | 1 | |

*The minus sign means down-regulation.
**1 means consistence between ADGE and regular microarray, while −1 means inconsistence.

ADGE Microarray Improves Accuracy of Detection

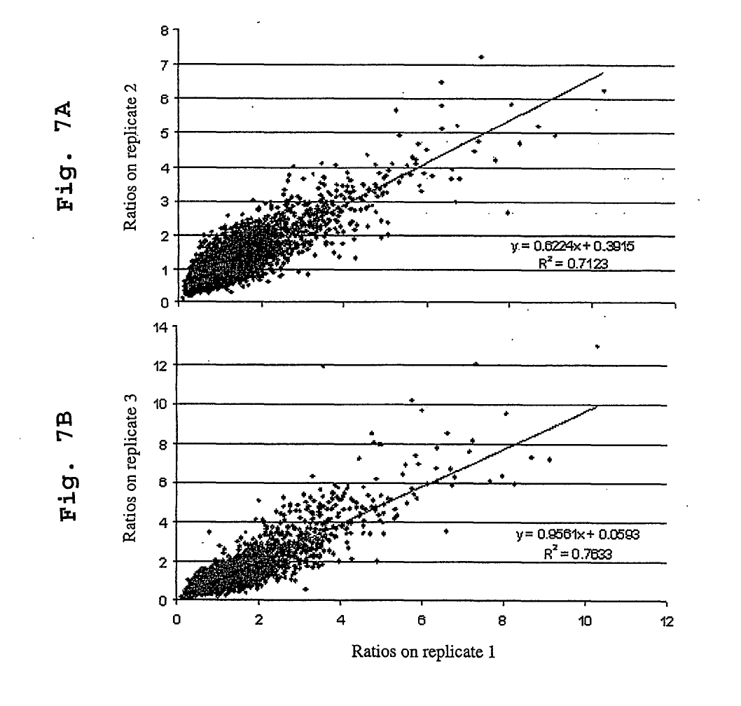

Multiple steps were integrated to improve accuracy of detection of gene expression profiles in the ADGE microarray method provided herein. The quadratic magnification increases the magnitude of expression ratios beyond the detection error observed when conventional microarray is used. The result of magnification allows one to raise the threshold for differential expression. PCR amplification of DNA templates enhances the signal intensities and reduces background. Direct incorporation of Cy dyes eliminates the variations of coupling efficiency. FIG. 7 shows the results of ADGE microarray were highly reproducible between replicates. The correlation coefficients between replicate 1 and replicate 2 and between replicate 1 and replicate 3 were 0.84 and 0.87, respectively. Thus, the detection results of the ADGE microarray method of the invention consistently reflected the altered expression of genes in the cell line. Unusual values of few genes in individual replicates were occasionally observed due to experimental error and were eliminated with statistical analysis.

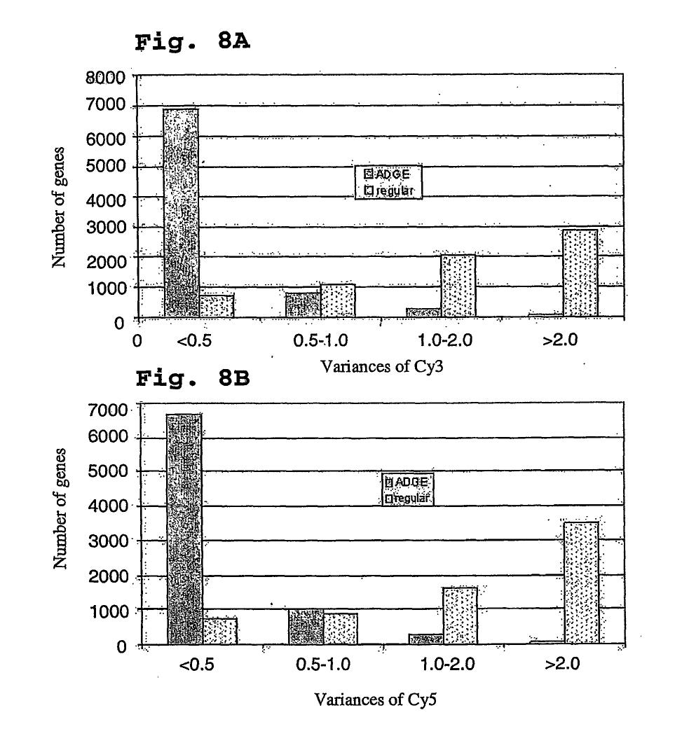

The variances of Cy3 and Cy5 intensities for each gene were less in the ADGE microarray method than in the conventional microarray method (FIG. 8). Most genes in ADGE microarray have variance of less than 0.5 in both Cy3 and Cy5 channels while most genes in regular microarray have variance of over 1.0. Thus, the results among replicates were more consistent, demonstrating less variation in ADGE microarray than in conventional microarray.

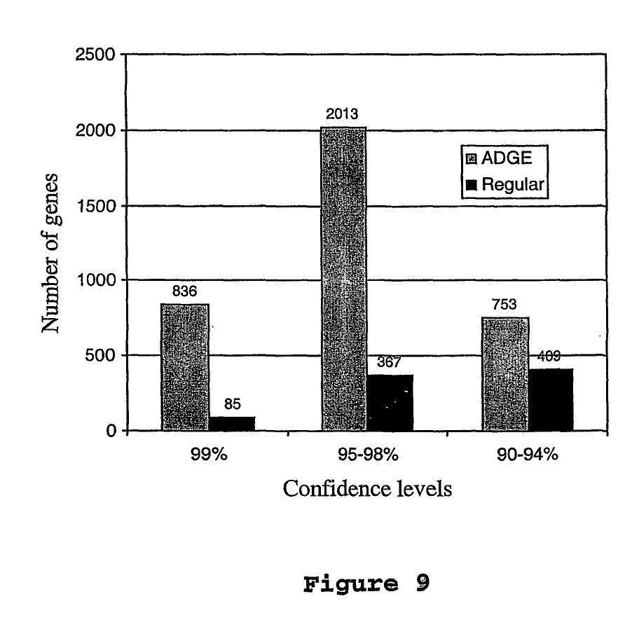

A greater number of genes showing significant changes were detected with high confidence levels in ADGE micraorray than in conventional microarray (FIG. 9). Using ADGE microarray, 836 genes were identified at the confidence level of 99%, 2013 genes at the level of 95-98%, and 753 genes at the level of 90-94%. In contrast, when conventional microarray methodology was employed, 85 genes were detected at the 99% level, 367 genes at the 95-98% level and 409 genes at the level of 90-94%. Thus the results of ADGE microarray were more reliable than those obtained using conventional microarray methods.

ADGE Microarray Enhances Signal and Requires Less Starting Material

The exponential amplification of PCR dramatically increases the amount of probes. The fluorescence intensity of probes can be further modulated by changing the ratio of Cy-dCTP and regular dCTP. In addition, both strands of probe DNA are labeled with Cy dye. The signal intensity can also be improved by coupling with other signal enhancement methods. The results provided herein demonstrate that ADGE microarray detected an average of 7983 genes among the three replicates with intensities of 200 or greater over the background while conventional microarray detected only an average of 6712 such genes. The strong signal in ADGE microarray also allows one to use smaller “Gain” value at the time of scanning, thus improving the ratios of signal to background.

ADGE Microarray Requires Less Starting Material.

Based on the current working protocol, 10 μg of total RNA from control and tester samples is used to generate 160 μl of reassociated DNA. 2 μl of the reassociated DNA is needed to make probe for one slide hybridization. Therefore, 125 ng of total RNA is required for one slide hybridization compared to 20 μg of total RNA for conventional microarray, a 160 fold difference. Therefore, the ADGE microarray method of the invention provides the advantage of requiring less starting material while at the same time producing a strong, reproducible signal.

ADGE Microarray Reveals Genes Associated with TLK 286 Drug Resistance

ADGE microarray was used to screen 20,000 genes in the TLK286 resistant cell line and its wild type. 472 genes with 4 fold or greater variations were selected. The result of analyzing these genes is shown in Table 2. 111 genes with the confidence level of 99% not only had a large magnitude of alteration but also were consistently detected across three replicates. Among the 111 genes, 44 genes are related to cell division and differentiation, kinase/phosphatase activities, and transcription regulation. It is suggested that 4 fold variation coupled with a confidence level of 99% is a reliable threshold for identifying genes which are differentially expressed. Another simple threshold is an average ratio of 4 or greater. 147 such genes were identified in the EL60/TLK286 cell line.

TABLE 4
Summary of screening 20,000 genes with ADGE microarray

| Confidence level | No of genes | average ratios | No of genes |
|---|---|---|---|
| 99% | 111 | >4 | 112 |
| 95-98% | 202 | 2˜4 | 168 |
| 90-94% | 80 | <2 | 5 |
| <90% | 79 | <−2 | 7 |
| | | −2˜−4 | 145 |
| | | >−4 | 35 |

The results of the ADGE microarray method were supported by previous studies regarding TLK286. A down-regulation of GST* and an overexpression of catalase were observed in the HL60/TLK286 cell line. GST* was detected as a decrease of 2.6-fold and catalase was detected as an increase of 2.7-fold with ADGE-microarray (Table 5). In addition, the expression of a, and 0 GST isoforms and microsomal GST was not found to be modified while the mRNA level of GST zeta was lower in resistant cells and GST M5 was in contrast higher in HL60/TLK286 (Table 5). The overexpression of GST M5 in HL60/TLK286 is consistent with the possible involvement of μisoforms in the resistance to alkylating agents (Horton et al, 1999). GST zeta is involved in the oxygenation of the hepatocarcinogen dichloroacetic acetic to glyoxylic acid and the isomerization of maleylacetoacetate to fumarylacetoacetate. Its role in anticancer drug resistance has not yet been assessed, however, because of its dowregulation in HL60/TLK286, this isoenzyme might be also involved in TLK286 activation.

TABLE 5
Genes of glutathione S-transferase family and kinase/phosphatase

| Gene ID | Accession ID folds* | t values | | confidence(%) |
|---|---|---|---|---|
| 245388-catalase | N54994 | 2.76 | 2.56 | 97.88 |
| 136235-glutathione S-transferase pi | R33642 | −2.67 | −4.85 | 99.69 |
| 823928-glutathione S-transferase theta 2 | AA490208 | −1.02 | −0.12 | 71.41 |
| 263014-glutathione S-transferase theta 1 | H99813 | 1.13 | 0.37 | 34.47 |
| 504791-glutathione S-transferase A4 | AA152347 | 1.37 | 0.85 | 94.45 |
| 256907-glutathione S-transferase A3 | N30096 | 1.14 | 0.39 | 82.06 |
| 82710-glutathione S-transferase A2 | T73468 | −1.06 | −0.46 | 45.50 |
| 277507-glutathione S-transferase M5 | N56898 | 2.11 | 2.25 | 99.73 |
| 768443-microsomal glutathione S-transferase 1 | AA495936 | 1.24 | 0.70 | 92.58 |
| 344432-microsomal glutathione S-transferase 2 | W73474 | −1.35 | −0.82 | 88.65 |
| 769676-glutathione transferase zeta 1 | AA428334 | −2.70 | −2.82 | 89.59 |
| 950445-protein phosphatase 2 (formerly 2A) | AA599092 | 5.25 | 5.70 | 99.32 |
| 301976-protein phosphatase 3 (formerly 2B) | N89721 | 3.66 | 6.08 | 99.70 |
| 509569-dual specificity phosphatase 10 | AA056608 | 3.67 | 4.06 | 99.95 |
| 1405689-PAK1 | AA890663 | 5.81 | 10.09 | 99.03 |
| 504877-MAPKKK5 | AA150828 | 3.59 | 10.30 | 99.42 |

*negative - down-regulated; positive - up-regulated.

The stress kinase c-Jun N-terminal kinase (JNK) but not the extracellular signal-regulated kinase (ERK) or p38 kinase was activated during TLK286 induced apoptosis. JNK is known to be activated and required in apoptosis induced by various stress stimuli. In HL60/TLK286, an overexpression of two phosphatases PP2 and MKP5 involved in JNK dephosphorylation and inactivation was detected (Table 5). PP2 is a serine/threonine phosphatase which dephosphorylates JNK during inflammatory cell signaling. MKP5 is a member of the dual specificity phosphatases which selectively dephosphorylates stress activated MAP kinases including JNK (Theodosiou et al, 1999). The overexpression of these two phosphatases might be partly responsible for the resistance of HL60/TLK86 by impairing the activity of JNK. In contrast the overexpression of Protein phosphatase 3 (PP3; N89721), p21/cdc42/Rac-activated kinase 1 (PAK1; AA890663) and Apoptosis signal-regulating idnase 1 (ASK1 or MAPKK5; AA150828), three proteins able to activate JNK pathway was also observed. PP3, also called calcineurin, is a calcium activated-enzyme which activates JNK and the transcription factor NFAT during T lymphocyte stimulation. ASK1 is activated during apotosis mediated by death receptor pathway and oxidative stress. ASK1 is inhibited by GST μ isoform, we demonstrated above that GST M5 was overexpressed in HL60/TLK286 and thus may inhibit ASK1 activity and/or activation. PAK1 which is activated by GTPase protein p21/cdc42/Rac1 is an activator of p38 kinase and JNK. The role of this protein in apoptosis appears to be ambiguous, PAK1 seems to be required for JNK-induced apoptosis following benzo(a)pyrene treatment, while it is also an antiapoptotic kinase which promotes cell survival by phophorylating Bad.

The gene expression profiles discussed above demonstrate that the ADGE microarray method of the invention provides sensitive, accurate and reliable information regarding the genes involved in regulation and maintenance of certain cellular states. The interpretation of this genetic signature is not only based on the magnitudes and directions of gene expression changes, but is also based on the biochemical interactions of these altered genes and the proteins they encode.

### Example III

### Different Applications of ADGE

ADGE integrates hybridization amplification and PCR amplification with uniquely designed adapters and primers in order to achieve the quadratic amplification of the expression ratios of genes in two samples. ADGE can be implemented in numerous ways. The implementation in Example I is appropriate for detecting a group of targeted genes. The implementation for coupling ADGE with microarray is appropriate for detecting all genes spotted on a chip. To facilitate the practice of each of these applications, kits are provided in accordance with the present invention. Three exemplary kits are as follows:

a) Targeted ADGE kits

For example:

p53-regulated ADGE kit

Purpose: monitoring transcript changes for a group of p53-regulated genes.

Components: CT and TT adapters, enzymes, buffers, a set of primers selected

from CT1-CT225 and TT1-TT225 specific to the p53-regulated targeted genes.

signal transduction ADGE kit

Purpose: monitoring transcript changes for a group of signal transduction genes.

Components: CT and TT adapters, enzymes, buffers, a set of primers selected from CT1-CT225 and TT1-TT225 specific to the signal transduction targeted genes.

b) Diagnostic ADGE kits

For example:

Skin cancer diagnostic ADGE kit

Purpose: monitoring transcript changes of a group of genes related to skin tumor development in order to determine the type and status of skin tumor.

Components: CT and TT adapters, enzymes, buffers, a set of primers selected

from CT1-CT225 and TT1-TT225 specific to the genes related to skin tumor.

c) Universal microarray-ADGE kit

Purpose: detecting transcript changes for all genes in a transcriptome from any organism, any tissue, any cell line.

Components: CT and TT adapters, enzymes, buffers, 4 CT primers and 4 TT primers with one selective nucleotide at 3′ ends, labeling elements (e.g. Cy3, Cy5).

The exemplary embodiments have been primarily described with reference to the figures which illustrate pertinent features of the embodiments. It should be appreciated that not all components or method steps of a complete implementation of a practical system are necessarily illustrated or described in detail. Rather, only those components or method steps necessary for a thorough understanding of the invention have been illustrated and described in detail. Actual implementations may utilize more steps or components or fewer steps or components. Thus, while certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for performing amplified differential gene expression, comprising:
a) providing a control nucleic acid sample and a tester nucleic acid sample;
b) fragmenting said control and tester nucleic acid samples;
c) ligating a first adapter molecule to said control nucleic acid sample and ligating a second adapter molecule to said tester nucleic acid sample;
d) subjecting said control and tester nucleic acid sample to conditions wherein hybridization between said control and tester nucleic acids samples occurs;
e) further subjecting said hybridized nucleic acid samples to a first polymerase chain reaction using a plurality of primers which are complementary to said first and second adapter molecules respectively;
f) isolating the amplified product from step e) and performing a secondary polymerase chain reaction;
g) separating products obtained from the polymerase chain reactions of step e) and step f) by gel electrophoresis; and
h) comparing the amplified products obtained from said control and tester nucleic acid samples to determine differences in gene expression profiles therein.

2. The method of claim 1, wherein said control and tester nucleic acid samples are obtained from reverse transcription of mRNA isolated from control and tester cellular samples.

3. The method of claim 1, wherein said control and tester nucleic acid samples are fragments using restriction enzyme digestion.

4. The method of claim 3, wherein said restriction enzyme is Taql.

5. The method of claim 1, wherein said control nucleic acid sample is isolated from a wild type cell and said tester nucleic acid sample is isolated from a diseased cell.

6. The method of claim 1, wherein said control nucleic acid sample is isolated from a wild type cell and said tester nucleic acid sample is isolated from a malignant cell.

7. The method of claim 1, wherein said control nucleic acid sample is isolated from a drug sensitive cell and said tester nucleic acid sample is isolated from a drug resistant cell.

8. A method for determining differences in gene expression profiles between control and tester nucleic acid samples comprising;
a) providing a control nucleic acid sample and a tester nucleic acid sample;
b) fragmenting said control and tester nucleic acid samples;
c) ligating a first adapter molecule to said control nucleic acid sample and ligating a second adapter molecule to said tester nucleic acid sample;
d) subjecting said control and tester nucleic acid sample to conditions wherein hybridization between said control and tester nucleic acids samples occurs;
e) further subjecting said hybridized nucleic acid samples to a first polymerase chain reaction using a plurality of primers which are complementary to said first and second adapter molecules respectively, in the presence of a first and second detectable label;
g) isolating products obtained from the polymerase chain reactions of step e);
h) denaturing said polymerase chain reaction products;
i) hybridizing said labeled, denatured products to a microarray chip comprising a multitude of target gene sequences;
j) determining the amount and identity of said nucleic acids in the control and tester samples as a function of position on said microarray chip, and presence of said detectable label; and
k) comparing the amplified products obtained from said control and tester nucleic acid samples to determine differences in gene expression profiles between said control and tester nucleic acid samples.

9. The method of claim 8, wherein said control and tester nucleic acid samples are obtained from reverse transcription of mRNA isolated from control and tester cellular samples.

10. The method of claim 8, wherein said control and tester nucleic acid samples are fragments using restriction enzyme digestion.

11. The method of claim 10, wherein said restriction enzyme is Taql.

12. The method of claim 8, wherein said first and second detectable labels are Cy3 and Cy5 and are separately incorporated into said control and tester nucleic acid respectively.

13. The method of claim 8, wherein said control nucleic acid sample is isolated from a wild type cell and said tester nucleic acid sample is isolated from a diseased cell.

14. The method of claim 8, wherein said control nucleic acid sample is isolated from a wild type cell and said tester nucleic acid sample is isolated from a malignant cell.

15. The method of claim 8, wherein said control nucleic acid sample is isolated from a drug sensitive cell and said tester nucleic acid sample is isolated from a drug resistant cell.
